(19) United States
(12) Patent Application Publication
(10) Pub. No.: US 2003/0144646 A1
(43) Pub. Date: Jul. 31, 2003

(54) Method and apparatus for collecting and transporting liquid

(76) Inventors: Erik Se, Ohlin (SE); Per Olsson, Stockholm (SE)

Correspondence Address: LARSON & TAYLOR, PLC, 1199 NORTH FAIRFAX STREET, SUITE 900, ALEXANDRIA, VA, 22314, US

(21) Appl. No.: 10/258,437

(22) PCT Filed: Apr. 24, 2001
(86) PCT No.: PCT/SE01/00868

(30) Foreign Application Priority Data
Apr. 28, 2000 (SE) 0001543-8

(51) Int. Cl. A61B 19/00; A61M 37/00; A61M 1/34

(52) U.S. Cl. 604/409; 422/44; 604/406; 604/6.15; 600/573

(57) ABSTRACT

The invention relates to a method and an apparatus for collecting and transporting liquids, for example blood, into one or more closed packages, for instance so called transfer bags (11, 12), or alternatively, directly transfering the blood into another liquid system, for instance a heart-lung machine, without any admixture of air or any other gas or gas mixture. The blood is collected without contact by human hand and without contamination of the blood and under sterile conditions. The apparatus comprises a suction nozzle (4) for sucking up the liquid or a liquid/air/gas mixture, a suction hose (5) for directing the liquid to a defoaming separator (7) for separating air from the liquid, and a recipient (8) in which the deacrated liquid is filtered and collected. When a certain volume has been collected, the liquid is directed without introducing air into said closed packages (11, 12) or communicated to another liquid system. The recipient (8) comprises a replaceable, flexible so-called canister bag (9) for collecting the liquid and arranged in a stiff, closed container (10) and provided with filters (17, 18) as well as filling and tapping conduits (22, 23). The recipient (8) and the corresponding packages (11, 12) are connected to the suction source (1) of the system for filling and tapping of the liquid.

The present invention relates to a method and an apparatus for collecting and transporting liquids of various types for an air-free or gas-free storage in one or more closed packages, for instance so-called transfer bags, or for transferring the liquid to another liquid system. The invention is specifically useful for handling of blood in connection with a surgical operation, but it can also be used when handling other types of liquids which may be easily damaged, oxidized etc. under the influence of air gas or the like, for example food stuff liquids, liquids which are corrosive or hazardous to the environment, such as oils, spillage liquids etc.

As the invention has been developed particularly in connection with the handling of blood, in the following it will mainly be described in connection with such handling.

There is always a short supply of blood and large amounts of blood are needed and used for blood transfusions, for instance in connection with surgical operations. Blood is a very expensive product, both with respect to the cost for collecting, but also with respect to the costs for testing and storing. During blood transfusions there is also a risque of transfer of jaundice, HIV infection and other diseases. In connection with a surgical operation the patient may lose large quantities of blood. This blood is normally wasted and the patient has to receive the corresponding amount of blood through transfusion, sometimes as much as several litres.

In order to solve the problem with blood supply it has been previously proposed to collect and intra operatively provide the patient with as much of his own blood as possible from the operation. The blood must be handled without contact by hand and under contamination free and sterile conditions. Autologous blood collecting devices are previously described in SE 467 725 and EP 0 742 737. According to these devices blood from the surgical operation is continuously sucked up by means of a suction hose and an anticoagulent agent is automatically added in proportion to the amount of sucked up blood. The blood is then caused, in a closed system, to pass suitable defoaming and separation filters and recipients to allow air bubbles included in the blood as well as undesirable particles to be separated from the blood. The blood is then collected in bottles or flexible bags entirely without any admixture of air or any other gas by means of a certain sub-pressure in the system. A suitable sub-pressure in the system can be achieved by connection to a negative pressure source, such as a vacuum pump.

The dosing of the anticoagulent agent is effectuated before the blood has reached the defoaming and filtering units. The dosage device is arranged to introduce the additive in an accurate proportion to the amount of the collected (sucked up) blood. This is achieved by providing a certain sub-pressure at different negative pressure levels to the dosage device and the suction nozzle for collecting the liquid. The pressure gradient between these different pressure levels is adjusted in such a way that no additive at all is introduced when there is no liquid (blood) in the suction nozzle, whereas the amount of dosage medium is almost completely in proportion to the amount of sucked liquid when liquid is sucked through the suction nozzle. Some examples of dosage devices are described in SE 467 725 and EP 0 742 737.

A previous separation and defoaming recipient comprises a closed container unit with a system of downwardly inclined planes on which the liquid is allowed to flow slowly, so that air bubbles which are included in the liquid has a possibility to escape to the upper part of the unit. Such a unit is described in SE 467 725. In the bottom of the recipient there is a valve which allows the liquid to be collected into two liquid containers, connected in parallel, and each comprising a flexible (blood)bag, a so-called transfer bag.

A similar de-air recipient is described in EP 0 742 737. Even in this case the recipient comprises a closed container unit 35 with a filter 36 for separating foam and a filter 37 for separating off particles, bone and tissue residuals which are eventually existing in the blood. A branch conduit 15 is arranged in the upper part of the container for evacuating air. In the bottom of the container there is a tapping tube 38 and a valve arrangement 39 for tapping liquid into two flexible bags 41, 42 arranged in a parallel relationship. An optical level detector is sensing the surface of the liquid in the container and initiates a closing of the bottom valve 39 before the liquid at the bottom of the container is completely emptied. This eliminates the risque that air enters into the flexible collecting bags.

In both of the cases which have been described now, the recipient comprises stiff container units, cans, which must be replaced and sterilized before each surgical operation. They must be big enough for about 2000 ml blood and they are clumsy to pack and handle.

An object of this invention is to provide an improved recipient arrangement which is more easy for sterilization, package and handling. A further object of the invention is to provide a recipient arrangement which allows the blood to be transferred substantially without any mechanical influence or actuation. Specifically, any mechanical valves or propelling means in the blood flow should be avoided.

According to the invention the recipient comprises a replaceable, flexible so-called canister bag which is arranged in a stiff container and provided with any necessary filters as well as filling and tapping conduits. Tapping of the blood into the transfer bags is effectuated by providing the recipient and the bags with an outer sub-pressure from the suction source of the system, which sub-pressure exceeds the internal suction pressure in the bags. This means that the bags are forced against the inner walls of its respective container.

According to a preferred embodiment the recipient comprises a first photo-optical detector for opening a tapping valve to a first transfer bag when the collected liquid has reached a specific level, and a second photo-optical detector for indicating a specific minimum level in the canister bag.

In the following the invention will be described more in detail with respect to the accompanying drawings, where

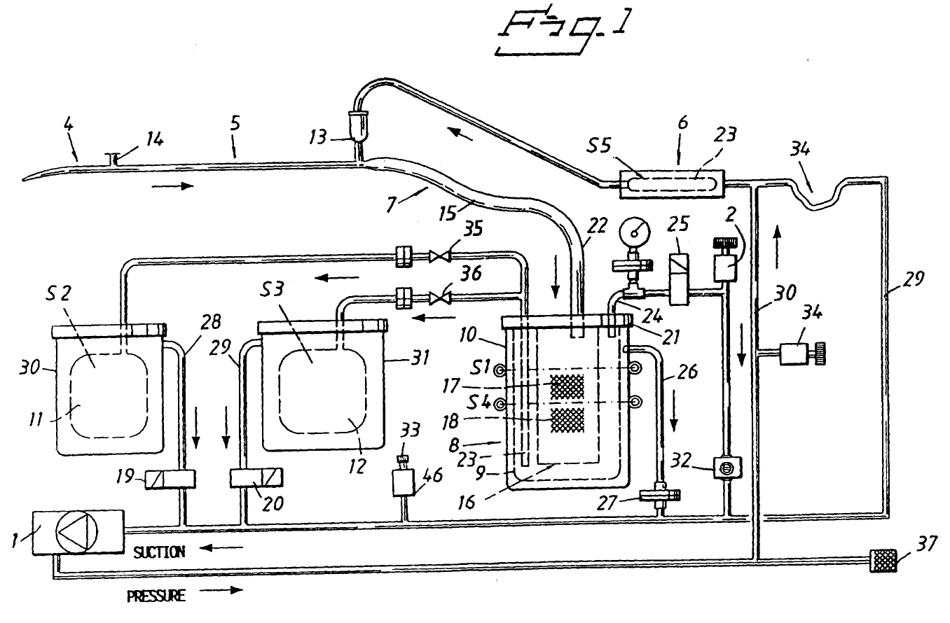

FIG. 1 shows schematically in the form of a flow chart a blood collecting system for a surgical operation and with a recipient according to the invention, and

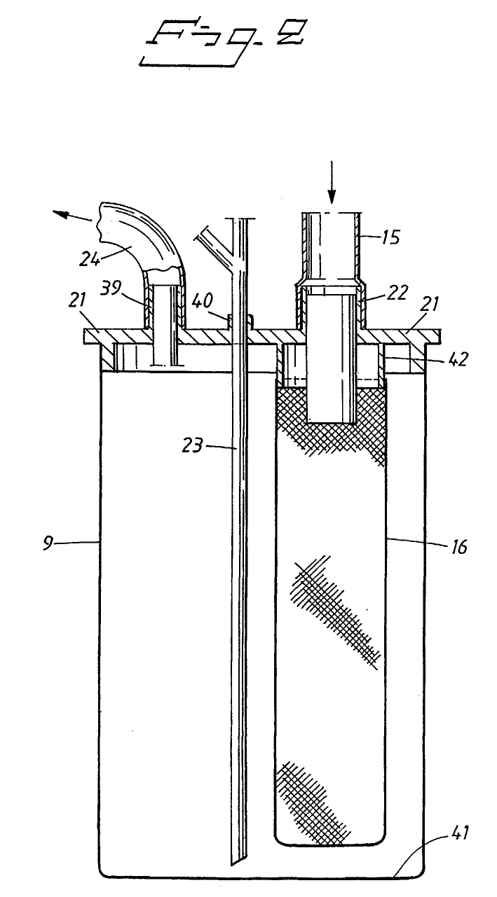

FIG. 2 shows an example of a canister bag with a lid for use in the recipient.

The blood collecting system according to FIG. 1 comprises a number of main parts which are operating with a certain predetermined pressure gradient in order to provide a sub-pressure (suction) in the system. The system comprises a vacuum pump 1 for creating a suitable sub-pressure. The sucking capacity of the pump could be approximately 2000 ml blood per minute, and the pump is preferably adjustable for different pressure levels by control means 2. As is well-known in the art, the pump has a suction side (suction) and a pressure side (pressure). The suction side of the pump is primarily connected to the system to provide the required sub-pressure in the system similar to the apparatus illustrated in EP 0 742 737. The pump is connected to the different parts in the system by means of a tube- or hose arrangement with valves, manometer and other pressure indicator means 3 which are known per se and therefore will not be described in any detail here. Also, sound silencing means 37 are arranged on the pressure side of the vacuum pump in order to reduce possible sound effects due to sudden pressure changes in the system.

The system comprises the following main parts: a suction nozzle 4 for sucking up blood at atmospheric pressure, a suction hose 5 connected to a unit 6 for dosing an agent in the form of an anticoagulent, a mixing and defoaming unit (mixer) 7 for admixing of the dosing agent into the blood and separating foam from the blood, a recipient 8 in the form of a canister bag 9 disposed in a stiff casing 10, and two transfer bags 11, 12 for an automatic collection of the blood.

This invention is specifically related to the recipient 8 and the filling of the transfer bags so the other parts in the system will not be described in any detail here. With respect to these parts it is also generally referred to the above-mentioned patent publications SE 467 725 and EP 0 742 737.

The dosing unit 6 is connected to the suction hose 5 at the inlet end of the mixer 7. The connection conduit is provided with a control and back valve arrangement with a drop chamber 13 for introducing anticoagulent in an accurate proportional, for instance 5-15%, to the amount of liquid. The basic level is infinitely adjustable and said proportional preset by using a specific cannula in the system or by means of the control valve. The dosage unit is described more in detail in our parallel patent application PCT/SE01/00870.

The blood is sucked up by means of the suction nozzle 4 which has a finger hole 14, and transferred via said suction hose 5 to the mixer which could be of the type shown in EP 0 742 737. The mixer has a mixing hose 15 which has a larger diameter than the suction hose 5 and which is rotated in the known way for admixing the dosing anticoagulent agent with the blood and separating air bubbles from the blood. The larger hose diameter is used all the way down into the recipient 8.

The centrifugated blood is then sucked via a filter arrangement 16, which in this case comprises a large filter 17 with a mask size of approximately 150-250 μm, for separating bone residuals and other particles from the blood, and a fine filter 18 with a mask size of approximately 30-40 μm, for separating coagulents, fat particles or the like, and collected in the canister bag 9 which may have a volume of about 2000 ml. A sensor S1, photo optical or capacitive, indicates when the collected blood has reached a volume of approximately 700 ml. Then a magnet valve 19 opens and approximately 500 ml blood is allowed to flow into the transfer bag 11. When this bag is full a sensor S2 gives a light and sound alarm signal to the control panel of the system for opening of the magnet valve 20 and closing the magnet valve 19. When the magnet valve 19 is closed the container 30 is connected to the atmospheric pressure (via an air inlet on the magnet valve 19) so that air is allowed to flow into the outer side of the bag 11 thereby eliminating the sub pressure outside the bag. This also facilitates the opening of the lid to the container 30, which lid otherwise should have been firmly fixed due to the sub-pressure in the container. Also the atmospheric pressure outside the bag 11 contributes to press out a possible air cushion formed in the upper part of the bag 11. Also the hose valve 35 is closed and instead the hose valve 36 is opened for filling of the transfer bag 12. When also this bag is full this is indicated by light or alarm signals in the same way by means of a sensor S3. The suction system can be used without interruption during the exchange of the transfer bags as the canister bag 9 has an extra capacity of minimum 1300 ml. Of course the transfer bags can be exchanged several times which makes the capacity of the apparatus substantially unlimited. Another photo optical sensor S4 in the recipient 8 indicates a certain minimum level in the canister bag 9.

The lid 21 of the canister bag has an inlet conduit 22 from the mixer 7, an outlet conduit 23 for tapping liquid and a suction conduit 24, which are all opening into the canister bag 9. The inlet conduit 22 and the suction conduit 24 are opening into the upper part of the bag, while the outlet conduit 23 is extending down into the bottom part of the bag.

The suction hose 24 is connected to the suction side of the vacuum pump via a shut-off valve 25, a manometer 3 and control means 2. The container 10 is also connected to the suction side of the vacuum pump 1, via a side hose 26 with filter 27. By this means the recipient is affected by two pressure levels, a first low pressure level P1 (large suction effect), which can be 250 mbar, by means of the side hose 26, and a second relatively higher pressure level P2 (lower suction effect) of about 150 mbar in the suction inlet of the canister bag, ie also in the inlet conduit 22, in the suction conduit 24 and in the outlet conduit 23. The first low pressure level PI (large suction effect) is sucking out, via the side hose 26, the canister bag against the inner wall of the container 10. The same pressure level P1 is also sucking out the transfer bags 11, 12 against their stiff outer casings 30, 31 when the magnet valves are opening. When the magnet valves then are closed the transfer bags are connected with the outer air and are affected by a slight suction backwards in order to remove possible air bubbles in the bags. The pressure difference P1-P2, of for instance 100 mbar, is large enough to provide a sufficient rapid flow in the system, but not so large that there is a risque that the blood cells are injured.

The apparatus blood flow passageways—such as suction nozzle, suction hose, mixer hose, filter arrangement and transfer system—has a layer of a blood compatible material as known in the art.

Unlike previous devices the magnet valves are not located in the blood flow but in the (air)connections 28, 29 to the suction side of the vacuum pump. When the magnet valves are opened the container with the transfer bags comes under full suction pressure P1 so that the transfer bags are fully opened for filling.

The suction pressure can be adjusted by means of control means 2. The hose system of the suction connection comprises a needle valve 32 and a safety valve 33. The pressure side of the vacuum pump is connected to the dosage device 6 via control means 34 and is described more in detail in our above-mentioned parallel patent application. According to said patent application the recipient 8 is only connected to the suction side and the tapping and filling cycles are effectuated entirely by the two (sub)pressure levels P1 and P2. The filter 27 prevents blood liquid from entering into the vacuum system of the apparatus in case of a broken canister bag from the start or broken during the process.

FIG. 2 shows an example of a canister bag 9. The bag can be made of a flexible plastic material of a type which is used within medical care for blood transfusions. The bag has a lid 21 welded onto the bag and provided with said inlet conduit 22 for connection to the mixer hose and a connection tube 39 for the suction hose 24. The lid has also a central opening 40 for the outlet conduit 23 which is extending down into the canister bag and stops close to the bottom 41 of the bag. The inlet conduit 22 has a diameter which corresponds to the large diameter mixer hose 15.

In connection to the inlet conduit 22 the lid has an internal sleeve 42 on which a filter arrangement 16 in the form of a hose made of a fine meshed fabric has been fastened so that the blood has to pass this hose before it is tapped out through the outlet hose 23. The filter arrangement with large and fine filters 17, 18 are welded together into one piece and glued onto the sleeve 42. Filters of this type are known per se and will not be described in any detail here. The sleeve 42 also has a shielding effect on the suction connection 39.

The invention is not limited to the example illustrated here but can be varied within the scope of the accompanying claims. Then, instead of filling transfer bags the apparatus can be used for blood supply to another liquid system, such as a heart/lung machine. Then the canister bag 9 is connected to the blood inlet of the heart/lung machine. In this case a certain over pressure is required for “pressing” the blood into the machine, which over pressure can be obtained by means of a pump known in the art, a roller pump or the like. Furthermore, in this case another sensor is required for indicating a minimum liquid level in the canister.

It should also be pointed out that the invention can be used post operatively, ie for collecting blood from a patient after a surgical operation.

1. Method for collecting and transporting liquids, for example blood, into one or more closed packages, for instance so called transfer bags (11, 12), or alternatively, directly transfering the blood into another liquid system, without any admixture of air or any other gas or gas mixture, without contact by human hand and without contamination of the blood and under sterile conditions, whereby the liquid or a liquid/air/gas mixture is sucked up by means of a suction nozzle (4) and transported through a suction hose (5) and a defoaming separator (7) for separating air from the liquid, to a recipient (8) in which the deaerated liquid is filtered and collected, whereupon the liquid, when a certain volume has been collected, without introducing air, is directed into said packages (11, 12) or communicated to another liquid system characterized by collecting the liquid in a replaceable, flexible so-called canister bag (9) which is arranged in a stiff, closed container (10) and provided with any necessary filters (17, 18) as well as filling and tapping conduits (22, 23) and providing the recipient (8) and the corresponding packages (11, 12) with a sub-pressure from the suction source (1) of the system for filling and tapping of the blood.

2. Method according to claim 1 characterized by connecting the suction source (1) to the container (10) of the recipient with a first relatively low pressure level P1 (strong suction effect) and to the flexible canister bag (9) with a second relatively higher pressure level P2 (lower suction effect).

3. Method according to claim 2 characterized by having a pressure difference between said pressure levels P1, P2 of about 100-150 mbar.

4. Method according to claim 3 characterized by connecting the container of the recipient to said first, relatively lower pressure level P1 (strong suction effect) so that the canister bag (9) is sucked out against the inner wall of the container (10) when filling the bag.

5. Method according to claim 2 characterized by arranging each of the transfer bags (11, 12) in a closed container (30, 31) and, when filling the bags, connecting the container to the first relatively low pressure level P1 (strong suction effect) via a connecting conduit (28, 29) with a valve (19, 20).

6. Method according to claim 5 characterized by sensing (S1) when the collected liquid in the canister bag (9) has reached a certain level and then opening the valve (19, 20) in the connecting conduit (28, 29) to the container (30, 31) for the transfer bag.

7. Method according to claim 1 characterized by adjusting the second, relatively higher pressure level P2 (lower suction effect) by control means (2).

8. Apparatus for collecting and transporting liquids, for example blood, into one or more closed packages, for instance so called transfer bags (11, 12), or alternatively, directly transfering the blood into another liquid system, without any admixture of air or any other gas or gas mixture, without contact by human hand and without contamination of the blood and under sterile conditions, comprising a suction nozzle (4) for sucking up the liquid or a liquid/air/gas mixture, a suction hose (5) for directing the liquid to a defoaming separator (7) for separating air from the liquid, and a recipient (8) in which the deaerated liquid is filtered and collected, whereupon the liquid, when a certain volume has been collected, without introducing air, is directed into said packages (11, 12) or communicated to another liquid system characterized in that the recipient (8) comprises a replaceable, flexible so-called canister bag (9) for collecting the liquid and arranged in a stiff, closed container (10) and provided with filters (17, 18) as well as filling and tapping conduits (22, 23) whereby the recipient (8) and the corresponding packages (11, 12) are connected to the suction source (1) of the system for filling and tapping of the liquid.

9. Apparatus according to claim 8 characterized in that the suction source (1) is connected to the container (10) of the recipient with a first relatively low pressure level P1 (strong suction effect) and to the flexible canister bag (9) with a second relatively higher pressure level P2 (lower suction effect).

10. Apparatus according to claim 9 characterized in that the pressure difference between the two pressure levels P1, P2 is about 100-150 mbar.

11. Apparatus according to claim 9 characterized in that each of the transfer bags (11, 12) are arranged in a closed container (30, 31) which container is connected to the suction source (1) with the first, relatively low pressure level P1 (strong suction effect) via a connecting conduit (28, 29) with a valve (19, 20).

12. Apparatus according to claim 9 characterized by a sensor (S1) for sensing when the collected liquid in the canister bag (9) has reached a certain level whereby the valve (19, 20) in the connecting conduit (28, 29) to the container (30, 31) for the transfer bag is opened.

13. Apparatus according to claim 9 characterized by control means (2) for adjusting the second, relatively higher pressure level P2 (lower suction effect).

14. Apparatus according to claim 8 characterized in that, in case the liquid consists of blood, the apparatus blood flow passageways—such as suction nozzle, suction hose, mixer hose, filter arrangement and transfer system—has a layer of a blood compatible material.

15. Apparatus according to claim 8 characterized by a further sensor for sensing a certain minimum liquid level in the recipient (8) when transferring the liquid into another liquid system.
